(19) United States
(12) Patent Application Publication
(10) Pub. No.: US 2019/0290710 A1
(43) Pub. Date: Sep. 26, 2019

(54) USE OF GLUCOCORTICOID ANALOGS TO ENHANCE RECOMBINANT ADENO-ASSOCIATED VIRUS YIELD

(71) Applicant: Dimension Therapeutics, Inc., Cambridge, MA (US)

(72) Inventors: Ying Jing, Wellesley, MA (US); Mingyang Jiang, Cambridge, MA (US); Kelly Reed Clark, Westerville, OH (US)

(21) Appl. No.: 16/316,426

(22) PCT Filed: Jul. 12, 2017
(86) PCT No.: PCT/US17/41738

§ 371 Date: Jan. 9, 2019

Related U.S. Application Data
(60) Provisional application No. 62/361,098, filed on Jul. 12, 2016.

(51) Int. Cl. A61K 35/761; C12N 15/86; C12N 5/09

(52) U.S. Cl. CPC A61K 35/761; C12N 15/86; C12N 2501/39; C12N 2750/14143; C12N 2750/14152; C12N 5/0693

(57) ABSTRACT

The invention provides methods for the production of recombinant adeno-associated virus vectors (rAAV), comprising contacting a host cell with a solution comprising a glucocorticoid analog, such as dexamethasone. Also provided are methods for increasing the production of rAAV by a host cell, comprising contacting a host cell with a solution comprising a glucocorticoid analog, such as dexamethasone.

### CROSS-REFERENCE TO RELATED APPLICATIONS

This application claims priority to and the benefit of U.S. Provisional patent application Ser. No. 62/361,098, filed on Jul. 12, 2016, and entitled “Use Of Glucocorticoid Analogs To Enhance Recombinant Adeno-Associated Virus Yield”, the entire disclosure of which is incorporated by reference herein.

### FIELD OF THE INVENTION

The invention relates generally to methods for enhancing recombinant adeno-associated virus vector (rAAV) yield, and, more particularly, the invention relates to the use of glucocorticoid analogs to enhance rAAV yield.

### BACKGROUND

Adeno-associated virus (AAV) is a non-pathogenic, replication-defective parvovirus. Recombinant AAV vectors (rAAV) have many unique features that make them attractive as vectors for gene therapy. In particular, rAAV vectors can deliver therapeutic genes to dividing and nondividing cells, and these genes can persist for extended periods without integrating into the genome of the targeted cell. Given the widespread therapeutic applications of rAAV, there exists an ongoing need for improved methods of rAAV vector production including methods to achieve high-titer rAAV vector yields. Previous attempts to improve the production of a variety of viral vectors have included the use of cell culture additives such as metals, trace supplements, salts, and others (See, e.g., Williams, J. Gen. Virol., 9(3): 251-5 (1970), Weinbauer et al., Limnology and Oceanography, 54(3): 774-784 (2009), Yang et al., Hepatology, 48(5): 1396-403 (2008), and U.S. Publication No. 20150353899).

### SUMMARY OF THE INVENTION

The invention is based, in part, upon the discovery that a host cell used in the production of recombinant adeno-associated virus vectors (rAAV) will produce increased amounts of rAAV when a glucocorticoid analog, such as dexamethasone, is added to the host cell culture. In one aspect, the invention provides a method for producing a recombinant adeno-associated virus vector (rAAV) comprising contacting a host cell with a solution comprising a glucocorticoid analog. In another aspect, the invention provides a method for increasing the amount of recombinant adeno-associated virus vector (rAAV) produced by a host cell, comprising contacting the host cell with a solution comprising a glucocorticoid analog. In one embodiment, the method further comprises the steps of harvesting and purifying the rAAV.

It is contemplated that the glucocorticoid analog may be dexamethasone, hydrocortisone, prednisolone, methylprednisolone, betamethasone, cortisone, prednisone, budesonide, and triamcinolone. In one embodiment, the glucocorticoid analog is dexamethasone.

It is contemplated that the concentration of glucocorticoid analog in solution may be greater than or equal to 1 μM, greater than or equal to 0.1 μM, greater than or equal to 0.01 μM, between 0 and 1 μM, between 0 and 0.1 μM, between 0 and 0.01 μM, between 0.01 and 1 μM, or between 0.01 and 0.1 μM. In one embodiment, the concentration of the glucocorticoid analog in the solution is sufficient to produce at least 1.5-fold greater quantities of secreted rAAV compared to that produced by a host cell not contacted with a solution comprising a glucocorticoid analog. In another embodiment, the concentration of the glucocorticoid analog in solution is sufficient to produce at least 1.5-fold greater quantities of total rAAV compared to that produced by a host cell not contacted with a solution comprising a glucocorticoid analog. In one embodiment, the host cell is contacted with the solution comprising the glucocorticoid analog for at least 2 days.

It is contemplated that the host cell may be a mammalian cell, for example, a HeLa, HEK293, COS, A549, or Vero cell. It is also contemplated that the host cell may be an insect cell, for example, a Sf9, Sf-21, Tn-368, or BTI-Tn-5B1-4. In one embodiment, the host cell is a HeLa cell. It is contemplated that host cell may comprise a heterologous nucleotide sequence flanked by AAV inverted terminal repeats, rep and cap genes, or helper virus genes. In one embodiment, the host cell comprises a heterologous nucleotide sequence flanked by AAV inverted terminal repeats, rep and cap genes, and helper virus genes.

In one embodiment, the host cell produces at least 1.5-fold greater quantities of secreted rAAV compared to that produced by a host cell not contacted with a solution comprising a glucocorticoid analog. In another embodiment, the host cell produces at least 1.5-fold greater quantities of total rAAV compared to that produced by a host cell not contacted with a solution comprising a glucocorticoid analog.

In other aspects, the invention provides a rAAV produced by any of the contemplated methods, a composition comprising a rAAV produced by any of the contemplated methods, or a composition comprising a host cell and a glucocorticoid analog.

These and other aspects and features of the invention are described in the following detailed description and claims.

### BRIEF DESCRIPTION OF THE DRAWINGS

The foregoing and other objects, features and advantages of the invention will become apparent from the following description of preferred embodiments, as illustrated in the accompanying drawings. Like referenced elements identify common features in the corresponding drawings.

FIG. 1 is a bar graph depicting the effect of dexamethasone (dex) on rAAV production from a HeLa producer cell line cultured in a shaker flask. Dexamethasone was added at the indicated concentrations and after 4 days rAAV yield (GC/ml) was measured by qPCR. Each value represents the mean of two independent experiments.

FIG. 2 is a bar graph depicting the effect of glucocorticoid analogs on rAAV production from a HeLa producer cell line cultured in a shaker flask. Hydrocortisone (hydr), predinosolone (pred) and dexamethasone (dex) were added at the indicated concentrations and after 4 days rAAV yield (GC/ml) was measured by qPCR. Each value represents the mean of two independent experiments.

FIG. 3 is a bar graph depicting the effect of dexamethasone (dex) on extracellular (top) and total (bottom) rAAV production from a HeLa producer cell line cultured in a 3 L bioreactor. Dexamethasone was added at the indicated concentration and rAAV yield (GC/ml) was measured by qPCR at the indicated time points. Each value represents the mean of two independent experiments.

FIG. 4 is a Simple Western Blot image depicting the effect of dexamethasone (dex) on Rep and Cap viral protein expression from a HeLa producer cell line on Days 2 and 3 (D2, D3) of culture in a 3 L bioreactor. Dexamethasone was added to the cell culture medium at a 1 μM concentration. Protein band images were generated by Compass software after running samples on the Wes capillary electrophoresis system (ProteinSimple) and using chromatogram output data.

FIG. 5 is a graph depicting the effect of dexamethasone on viral genome amplification over the course of three days after induction of a HeLa producer cell line cultured in a 3 L bioreactor. Dexamethasone was added to the cell culture medium at a 1 μM concentration. Viral genome copy amplification (GC/cell) was measured by qPCR at the indicated time points. Each value was normalized to Day 0 GC/cell for a fold-change comparison.

### DETAILED DESCRIPTION

The invention is based, in part, upon the discovery that a host cell used in the production of recombinant adeno-associated virus vectors (rAAV) will produce increased amounts of rAAV when a glucocorticoid analog, such as dexamethasone, is added to the host cell culture. In one aspect, the invention provides a method for producing rAAV comprising contacting a host cell with a solution comprising a glucocorticoid analog. In another aspect, the invention provides a method for increasing the amount of recombinant adeno-associated virus vector (rAAV) produced by a host cell, comprising contacting the host cell with a solution comprising a glucocorticoid analog. In another aspect, the invention provides a method for increasing the amount of Rep and/or Cap protein produced by a host cell, comprising contacting the host cell with a solution comprising a glucocorticoid analog. In another aspect, the invention provides a method for increasing viral genome amplification by a host cell, comprising contacting the host cell with a solution comprising a glucocorticoid analog.

### I. Adeno-Associated Virus

Adeno-associated virus (AAV) is a small, nonenveloped icosahedral virus of the genus Dependoparvovirus and family Parvovirus. AAV has a single-stranded linear DNA genome of approximately 4.7 kb. AAV includes numerous serologically distinguishable types including serotypes AAV-1 to AAV-12, as well as more than 100 serotypes from nonhuman primates (See, e.g., Srivastava, J. Cell Biochem., 105(1): 17-24 (2008), and Gao et al., J. Virol., 78(12), 6381-6388 (2004)). Any AAV type may be used in the methods of the present invention. AAV is capable of infecting both dividing and quiescent cells of several tissue types, with different AAV serotypes exhibiting different tissue tropism. AAV is non-autonomously replicating, and has a life cycle with a latent phase and an infectious phase. In the latent phase, after a cell is infected with an AAV, the AAV site-specifically integrates into the host's genome as a provirus. The infectious phase does not occur unless the cell is also infected with a helper virus (for example, adenovirus (AV) or herpes simplex virus), which allows the AAV to replicate.

The wild-type AAV genome contains two 145 nucleotide inverted terminal repeats (ITRs), which contain signal sequences directing AAV replication, genome encapsidation and integration. In addition to the ITRs, three AAV promoters, p5, p19, and p40, drive expression of two open reading frames encoding rep and cap genes. Two rep promoters, coupled with differential splicing of the single AAV intron, result in the production of four rep proteins (Rep 78, Rep 68, Rep 52, and Rep 40) from the rep gene. Rep proteins are responsible for genomic replication. The Cap gene is expressed from the p40 promoter, and encodes three capsid proteins (VP1, VP2, and VP3) which are splice variants of the cap gene. These proteins form the capsid of the AAV particle.

Because the cis-acting signals for replication, encapsidation, and integration are contained within the ITRs, some or all of the 4.3 kb internal genome may be replaced with foreign DNA, for example, an expression cassette for an exogenous protein of interest. In this case the rep and cap proteins are provided in trans on, for example, a plasmid. In order to produce an AAV vector, a host cell line permissive of AAV replication must express the rep and cap genes, the ITR-flanked expression cassette, and helper functions provided by a helper virus, for example AV genes E1a, E1b55K, E2a, E4orf6, and VA (Weitzman et al., Adeno-associated virus biology. Adeno-Associated Virus: Methods and Protocols, pp. 1-23, 2011). Production of AAV vector can also result in the production of helper virus particles, which must be removed or inactivated prior to use of the AAV vector. Numerous cell types are suitable for producing AAV vectors, including HEK293 cells, COS cells, HeLa cells, BHK cells, Vero cells, as well as insect cells (See e.g. U.S. Pat. Nos. 6,156,303, 5,387,484, 5,741,683, 5,691,176, 5,688,676, 8,163,543, U.S. Publication No. 20020081721, PCT Publication Nos. WO00/47757, WO00/24916, and WO96/17947). AAV vectors are typically produced in these cell types by one plasmid containing the ITR-flanked expression cassette, and one or more additional plasmids providing the additional AAV and helper virus genes.

AAV of any serotype may be used in the present invention. Similarly, it is contemplated that any AV type may be used, and a person of skill in the art will be able to identify AAV and AV types suitable for the production of their desired recombinant AAV vector (rAAV). AAV and AV particles may be purified, for example by affinity chromatography, iodixonal gradient, or CsCl gradient.

The genome of wild-type AAV is single-stranded DNA and is 4.7 kb. AAV vectors may have single-stranded genomes that are 4.7 kb in size, or are larger or smaller than 4.7 kb, including oversized genomes that are as large as 5.2 kb, or as small as 3.0 kb. Further, vector genomes may be substantially self-complementary, so that within the virus the genome is substantially double stranded. AAV vectors containing genomes of all types are suitable for use in the method of the instant invention.

As discussed above, AAV requires co-infection with a helper virus in order to enter the infectious phase of its life cycle. Helper viruses include Adenovirus (AV), and herpes simplex virus (HSV), and systems exist for producing AAV in insect cells using baculovirus. It has also been proposed that papilloma viruses may also provide a helper function for AAV (See, e.g., Hermonat et al., Molecular Therapy 9, S289-S290 (2004)). Helper viruses include any virus capable of creating an allowing AAV replication. AV is a nonenveloped nuclear DNA virus with a double-stranded DNA genome of approximately 36 kb. AV is capable of rescuing latent AAV provirus in a cell, by providing E1a, E1b55K, E2a, E4orf6, and VA genes, allowing AAV replication and encapsidation. HSV is a family of viruses that have a relatively large double-stranded linear DNA genome encapsidated in an icosahedral capsid, which is wrapped in a lipid bilayer envelope. HSV are infectious and highly transmissible. The following HSV-1 replication proteins were identified as necessary for AAV replication: the helicase/primase complex (UL5, UL8, and UL52) and the DNA binding protein ICP8 encoded by the UL29 gene, with other proteins enhancing the helper function.

2. Production of rAAV

The present invention comprises the production of a recombinant adeno-associated virus vector (rAAV) from a host cell, using any suitable method known in the art. As used herein, the term “host cell” refers to any cell or cells capable of producing a rAAV. In some embodiments, the host cell is a mammalian cell, for example, a HeLa cell, COS cell, HEK293 cell, A549 cell, BHK cell, or Vero cell. In other embodiments, the host cell is an insect cell, for example, a Sf9 cell, Sf-21 cell, Tn-368 cell, or BTI-Tn-5B1-4 (High-Five) cell. Unless otherwise indicated, the terms “cell” or “cell line” are understood to include modified or engineered variants of the indicated cell or cell line.

As discussed above, to allow for production of rAAV, the host cell must be provided with AAV inverted terminal repeats (ITRs), which may, for example, flank a heterologous nucleotide sequence of interest, AAV rep and cap gene functions, as well as additional helper functions. These may be provided to the host cell using any number of appropriate plasmids or vectors. Additional helper functions can be provided by, for example, an adenovirus (AV) infection, by a plasmid that carries all of the required AV helper function genes, or by other viruses such as HSV or baculovirus. Any genes, gene functions, or other genetic material necessary for rAAV production by the host cell may transiently exist within the host cell, or be stably inserted into the host cell genome. In some embodiments, the host cell is a producer cell comprising AAV rep and cap gene functions and a rAAV vector genome. In some embodiments, the host cell is a packaging cell comprising AAV rep and cap gene functions which at the time of production is provided a rAAV vector genome by a separate recombinant virus. rAAV production methods suitable for use with the methods of the current invention include those disclosed in Clark et al., Human Gene Therapy 6:1329-1341 (1995), Martin et al., Human Gene Therapy Methods 24:253-269 (2013), Thorne et al., Human Gene Therapy 20:707-714 (2009), Fraser Wright, Human Gene Therapy 20:698-706 (2009), and Virag et al., Human Gene Therapy 20:807-817 (2009).

### 3. Glucocorticoid Analogs

Glucocorticoids are a class of steroid hormones with important roles in physiological processes including immune responses, stress responses, and metabolism. Glucocorticoids bind to and effect function through glucocorticoid receptors, and have been used therapeutically as anti-inflammatory agents. Descriptions of glucocorticoid analogs include those disclosed in Buckbinder et al., Current Drug Targets-Inflammation and Allergy 1:127-136 (2002) and Reeves et al., Endocrine, Metabolic & Immune Disorders-Drug Targets 12(1):95-103 (2012). As used herein, the term “glucocorticoid analog” includes natural or synthetic glucocorticoids and variants thereof. Exemplary glucocorticoid analogs include dexamethasone, hydrocortisone, prednisolone, methylprednisolone, betamethasone, cortisone, prednisone, budesonide, and triamcinolone.

In the present invention, a host cell capable of producing rAAV is contacted with a solution comprising a glucocorticoid analog. The host cell may be contacted with the solution comprising the glucocorticoid analog by any appropriate means, and any appropriate time during the rAAV production process. In some embodiments, the glucocorticoid analog is dissolved in a suitable solvent, such as DMSO, and the resulting glucocorticoid analog solution is added to a cell culture. In some embodiments, the glucocorticoid analog in a powder form is added directly to a cell culture. In some embodiments, the glucocorticoid analog is added to a cell culture medium prior to use of the medium in culturing of the host cell. In yet another embodiment, the glucocorticoid analog is added to the host cell by use of a feed or bolus shot.

In some embodiments the final concentration of the glucocorticoid analog in the solution contacted with the host cell is greater than or equal to 1 μM, in some embodiments greater than or equal to 0.1 μM, in some embodiments greater than or equal to 0.01 μM. In some embodiments the final concentration of the glucocorticoid analog in solution is between 0 and 1 μM, in some embodiments between 0 and 0.1 μM, in some embodiments between 0 and 0.01 μM, in some embodiments between 0.01 and 1 μM, and in some embodiments between 0.01 and 0.1 μM. In some embodiments, the final concentration of the glucocorticoid analog in solution is the concentration sufficient to produce a desired increased in yield. For example, in some embodiments, the final concentration of the glucocorticoid analog in the solution is sufficient to produce at least 1.5-fold greater quantities of secreted rAAV compared to that produced by a host cell not contacted with a solution comprising a glucocorticoid analog, and in some embodiments, the final concentration of the glucocorticoid analog in solution is sufficient to produce at least 1.5-fold greater quantities of total rAAV compared to that produced by a host cell not contacted with a solution comprising a glucocorticoid analog.

4. Purification of rAAV Particles

In some embodiments of the current invention, the rAAV particles are harvested and/or purified from the host cell after the host cell has been contacted with the solution comprising the glucocorticoid analog. rAAV particles may be obtained from host cells by lysing the cells. Lysis of host cells can be accomplished by methods that chemically or enzymatically treat the cells in order to release infections viral particles. These methods include the use of nucleases such as benzonase or DNAse, proteases such as trypsin, or detergents or surfactants. Physical disruption, such as homogenization or grinding, or the application of pressure via a microfluidizer pressure cell, or freeze-thaw cycles may also be used. Alternatively, supernatant may be collected from host cells without the need for cell lysis. As used herein, “total rAAV” refers to the total rAAV produced by a host cell, and “secreted rAAV” refers to rAAV that can be can be harvested from a host cell without the need to cell lysis.

After harvesting rAAV particles, it may be necessary to purify the sample containing rAAV, to remove, for example, the cellular debris resulting from cell lysis. Methods of minimal purification of AAV particles are known in the art. Two exemplary purification methods are Cesium chloride (CsCl)- and iodixanol-based density gradient purification. Both methods are described in Strobel et al., Human Gene Therapy Methods., 26(4): 147-157 (2015). Minimal purification can also be accomplished using affinity chromatography using, for example AVB Sepharose affinity resin (GE Healthcare Bio-Sciences AB, Uppsala, Sweden). Methods of AAV purification using AVB Sepharose affinity resin are described in, for example, Wang et al., Mol Ther Methods Clin Dev., 2:15040 (2015). Following purification, rAAV particles may be filtered and stored at ≤−60° C.

5. Quantification of rAAV Particles

Quantification of rAAV particles is complicated by the fact that AAV infection does not result in cytopathic effect in vitro, and therefore plaque assays cannot be used to determine infectious titers. rAAV particles can be quantified using a number of methods, however, including quantitative polymerase chain reaction (qPCR) (Clark et al., Hum. Gene Ther. 10, 1031-1039 (1999)) or dot-blot hybridization (Samulski et al., J. Virol. 63, 3822-3828 (1989)), or by optical density of highly purified vector preparations (Sommer et al., Mol. Ther. 7, 122-128 (2003)). DNase-resistant particles (DRP) can be quantified by real-time quantitative polymerase chain reaction (qPCR) (DRP-qPCR) in a thermocycler (for example, an iCycler iQ 96-well block format thermocycler (Bio-Rad, Hercules, Calif.)). Samples containing rAAV particles are incubated in the presence of DNase I (100 U/ml; Promega, Madison, Wis.) at 37° C. for 60 min, followed by proteinase K (Invitrogen, Carlsbad, Calif.) digestion (10 U/ml) at 50° C. for 60 min, and then denatured at 95° C. for 30 min. The primer-probe set used should be specific to a non-native portion of the rAAV vector genome, for example, the poly(A) sequence of the protein of interest. The PCR product can be amplified using any appropriate set of cycling parameters, based on the length and composition of the primers, probe, and amplified sequence. Alternative protocols are disclosed in, for example, Lock et al., Human Gene Therapy Methods 25(2): 115-125 (2014).

Viral genome amplification can also be measured using qPCR techniques similar to those described above. However, in order to quantify total genome amplification within producer cells, only intracellular samples are collected and the samples are not treated with DNase I in order to measure both packaged and unpackaged viral genomes. Viral genome amplification may be calculated on a per-host-cell basis by concomitantly measuring a host cell housekeeping gene, for example, RNase P.

The infectivity of rAAV particles can be determined using a TCID50 (tissue culture infectious dose at 50%) assay, as described for example in Zhen et al., Human Gene Therapy 15:709-715 (2004). In this assay, rAAV vector particles are serially diluted and used to co-infect a Rep/Cap-expressing cell line along with AV particles in 96-well plates. 48 hours post-infection, total cellular DNA from infected and control wells is extracted. rAAV vector replication is then measured using qPCR with transgene-specific probe and primers. TCID50 infectivity per milliliter (TCID50/ml) is calculated with the Kärber equation, using the ratios of wells positive for AAV at 10-fold serial dilutions.

Throughout the description, where apparatus, devices, and systems are described as having, including, or comprising specific components, or where processes and methods are described as having, including, or comprising specific steps, it is contemplated that, additionally, there are apparatus, devices, and systems of the present invention that consist essentially of, or consist of, the recited components, and that there are processes and methods according to the present invention that consist essentially of, or consist of, the recited processing steps.

Practice of the invention will be more fully understood from the foregoing examples, which are presented herein for illustrative purposes only, and should not be construed as limiting the invention in any way.

### EXAMPLES

### Example 1

A number of cell culture additives including metals, trace supplements, salts, and others, were tested for their ability to increase production of recombinant adeno-associated virus vector (rAAV) by a HeLa host cell line. No or minimal impact on yield was observed following an increase of iron concentration in the culture from 0.1 mg/L to 10 mg/L, or an increase of copper concentration from 0.1 μM to 10 μM. A minimal impact on yield was observed following supplementation of the culture with 0.19 g/L of calcium, with higher calcium concentrations causing media precipitation. A slightly negative impact on yield was observed following an increase of magnesium concentration from 1 mg/L to 30 mg/L. No or minimal impact on yield was observed following an increase in concentration of a fatty acid supplement (Fatty Acid Supplement, Sigma-Aldrich F7050) in the culture from 0.2 mL/L to 2.5 mL/L, or an increase in concentration of a nucleoside mixture (EmbryoMax® Nucleosides (100×), EMD Millipore ES-008-D) from 0.03× to 3×. Supplementation of the culture with lipid mixtures (Chemically Defined Lipid Concentrate, Gibco 11905-031; Lipid mixture 1, Sigma-Aldrich, L0288) resulted in no or minimal impact on yield. No impact was also observed following supplementation of the culture with up to 6 mM phosphate salt, with higher than 9 mM phosphate salt causing media precipitation. Both an increase of putrescine concentration from 0.1 mM to 10 mM and an increase in spermine concentration from 0.1 mM to 10 mM had a negative effect on yield. No or minimal impact was observed with up to 0.9 g/L of Pluronic. However, as described in Examples 2 and 3, supplementation with the glucocorticoid analogs dexamethasone, hydrocortisone, and prednisolone resulted in increased rAAV yield.

### Example 2

This example describes increased production of rAAV by a host cell cultured in shaker flasks following the addition of dexamethasone to the cell culture. The HeLa host cell line was engineered from HeLa S3 parental cells. In this system, a single plasmid containing three components: the vector sequence, the AAV rep and cap genes and a selectable marker gene is stably transfected into HeLaS3 cells. The cells were cultured in a protein-free, chemically-defined production medium with a fraction of growth medium carried over through inoculation. The cells were cultured in 250 mL shaker flasks with starting volumes of 100 mL and initial cell densities of 1×10⁶ cells/mL, and maintained at 37° C. and 5% CO2. The cultures were sampled daily to monitor cell growth and metabolites and the pH was adjusted as needed using 1M sodium carbonate.

Stock solutions of dexamethasone (dex) dissolved in DMSO were added to the cell culture to final concentrations of 0-1 μM. After 4 days, rAAV yield was determined by measuring both the secreted (extracellular) and total rAAV in the cell culture harvest by qPCR.

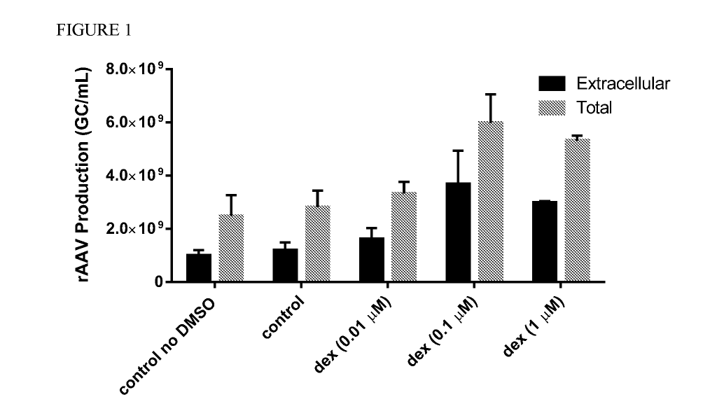

As depicted in FIG. 1, dexamethasone increased both the extracellular and total rAAV yield of a HeLa host cell line, at concentrations as low as 0.01 μM.

### Example 3

This example describes increased production of rAAV by a host cell cultured in shaker flasks following the addition of the glucocorticoid analogs dexamethasone, prednisolone, and hydrocortisone to the cell culture. A HeLa host cell line engineered from HeLa S3 parental cells was cultured in a protein-free, chemically-defined production medium with a fraction of growth medium carried over through inoculation. The cells were cultured in 250 mL shaker flasks with starting volumes of 100 mL and initial cell densities of 0.7×10⁶ cells/mL, and maintained at 37° C. and 5% CO2. The cultures were sampled daily to monitor cell growth and metabolites and the pH was adjusted as needed using 1M sodium carbonate.

Stock solutions of dexamethasone (dex), prednisolone (pred), and hydrocortisone (hydr) dissolved in DMSO were added to the cell culture to final concentrations of 0.1 and 1 μM. After 4 days, rAAV yield was determined by measuring both the secreted (extracellular) and total rAAV in the cell culture harvest by qPCR.

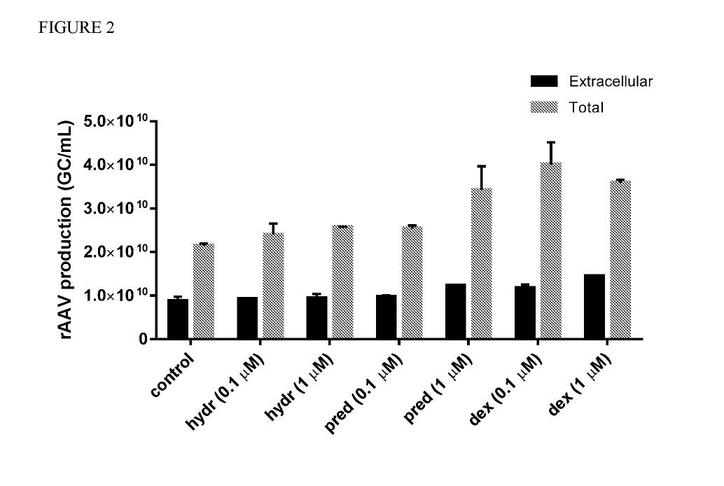

As depicted in FIG. 2, increased rAAV yield was observed after addition of prednisolone and hydrocortisone in addition to dexamethasone. These results demonstrate that the beneficial effect of dexamethasone on rAAV production could be replicated with other glucocorticoid analogs.

### Example 4

This example describes increased production of rAAV by a host cell cultured in a 3 L bioreactor following the addition of dexamethasone to the cell culture. The HeLa host cell line was engineered from HeLa S3 parental cells. The cells were cultured in a protein-free, chemically-defined production medium with a fraction of growth medium carried over through inoculation. The cells were cultured in 3 L bioreactors with starting volumes of 2 L and initial cell densities of 0.6×10⁶ cells/mL, and maintained at temperature and dissolved oxygen (DO) set points of 37° C. and 50%, respectively, for five days. pH was controlled using 1M sodium carbonate at set point 7.3 for four days and elevated to 8.0 for the remainder of the experiment. The cultures were sampled daily to monitor cell growth and metabolites.

Stock solutions of dexamethasone (dex) dissolved in DMSO were added to the cell culture to a final concentration of 1 μM. After 3, 4, and 5 days, rAAV yield was determined by measuring both the secreted (extracellular) and total rAAV in the cell culture harvest by qPCR.

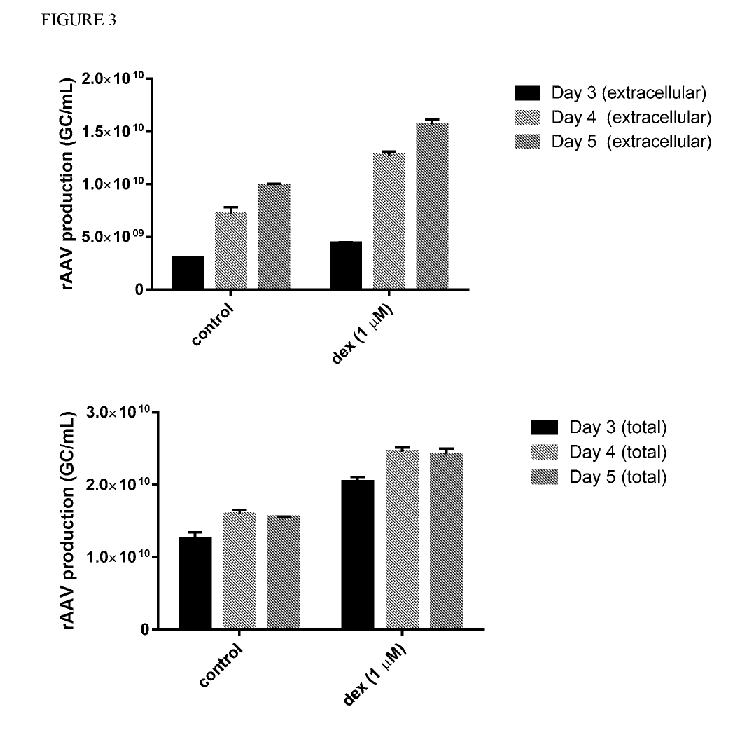

As depicted in FIG. 3, dexamethasone increased both the extracellular and total rAAV yield of a HeLa host cell line cultured in a 3 L bioreactor, similarly to that observed for a HeLa host cell line cultured in shaker flasks. These results demonstrate that the beneficial effect of dexamethasone on rAAV production could be replicated in different production contexts.

### Example 5

This example describes increased production of Rep and Cap viral proteins by a host cell cultured in a 3 L bioreactor following the addition of dexamethasone to the cell culture medium. The HeLa host cell line was engineered from HeLa S3 parental cells. The cells were cultured in a protein-free, chemically-defined production medium with a fraction of growth medium carried over through inoculation. The cells were cultured in 3 L bioreactors with starting volumes of 2 L and initial cell densities of 0.7×10⁶ cells/mL, and maintained at temperature and dissolved oxygen (DO) set points of 37° C. and 50%, respectively, for four days. pH was controlled using 1M sodium carbonate at set point 7.3 for the entire experiment. The cultures were sampled daily to monitor cell growth and viability.

Stock solutions of dexamethasone (dex) dissolved in DMSO were added to the cell culture medium to a final concentration of 1 μM. After 2 and 3 days, protein expression was visualized by collecting cell culture samples and running a Simple Western Immunoassay using a Wes Capillary Electrophoresis System (ProteinSimple) according to the manufacturer's instructions using mouse anti-AAV Replicase antibody clone 303.9 (1:50 dilution) (American Research Products), and mouse anti-AAV VP1/VP2/VP3 antibody B1 (1:400 dilution) (American Research Products), with mouse anti-vinculin antibody used as a loading control.

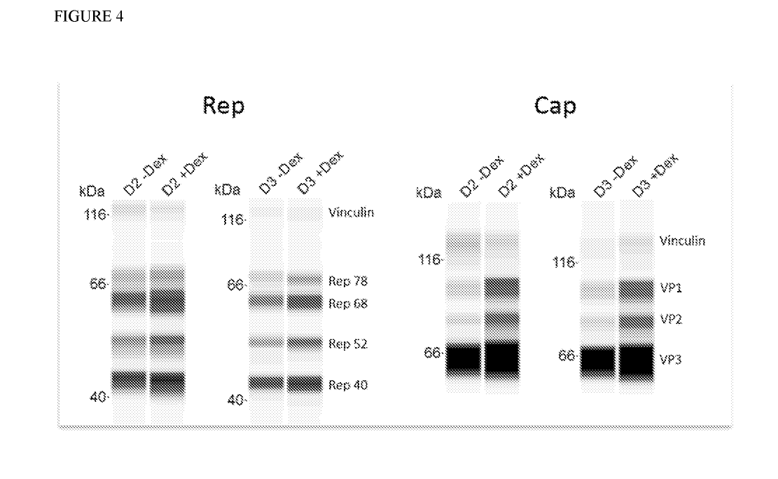

As depicted in FIG. 4, dexamethasone increased both Rep and Cap viral protein production from a HeLa host cell line cultured in a 3 L bioreactor. These results demonstrate that dexamethasone has a beneficial effect on the production of important viral proteins, likely facilitating the observed increase in virus production.

### Example 6

This example describes increased viral genome amplification by a host cell cultured in a 3 L bioreactor following the addition of dexamethasone to the cell culture. The HeLa host cell line was engineered from HeLa S3 parental cells. The cells were cultured in a protein-free, chemically-defined production medium with a fraction of growth medium carried over through inoculation. The cells were cultured in 3 L bioreactors with starting volumes of 2 L and initial cell densities of 0.7×10⁶ cells/mL, and maintained at temperature and dissolved oxygen (DO) set points of 37° C. and 50%, respectively, for four days. pH was controlled using 1M sodium carbonate at set point 7.3 for the entire experiment. The cultures were sampled daily to monitor cell growth and viability.

Stock solutions of dexamethasone (dex) dissolved in DMSO were added to the cell culture medium to a final concentration of 1 μM. After 0, 1, 2, and 3 days, intracellular culture samples were collected and viral genome amplification was measured by qPCR.

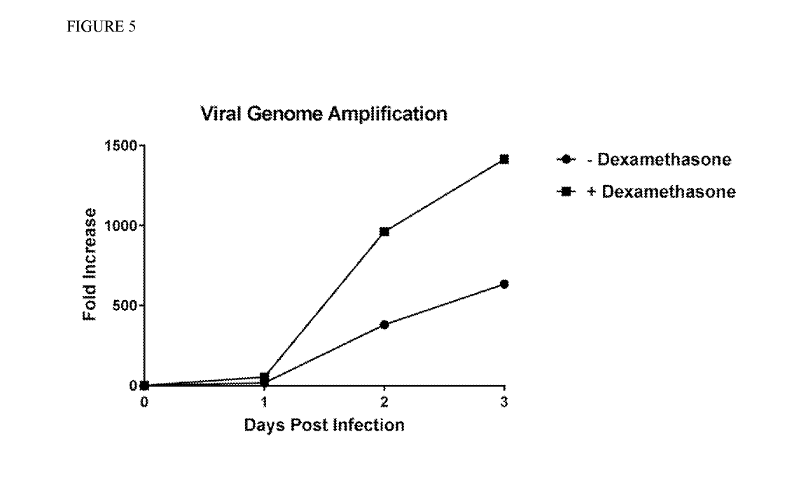

As depicted in FIG. 5, dexamethasone increased viral genome amplification from a HeLa host cell line cultured in a 3 L bioreactor. These results demonstrate that dexamethasone has a beneficial effect on the replication of viral genomes, likely facilitating the observed increase in virus production.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A method for producing a recombinant adeno-associated virus vector (rAAV) comprising contacting a host cell with a solution comprising a glucocorticoid analog.

2. A method for increasing the amount of recombinant adeno-associated virus vector (rAAV) produced by a host cell, comprising contacting the host cell with a solution comprising a glucocorticoid analog.

3. The method of claim 1 or 2 wherein the glucocorticoid analog is selected from the group consisting of dexamethasone, hydrocortisone, prednisolone, methylprednisolone, betamethasone, cortisone, prednisone, budesonide, and triamcinolone.

4. The method of claim 3, wherein the glucocorticoid analog is dexamethasone.

5. The method of any one of claims 1-4, wherein the final concentration of the glucocorticoid analog in the solution is greater than or equal to 1 μM.

6. The method of any one of claims 1-4, wherein the final concentration of the glucocorticoid analog in the solution is greater than or equal to 0.1 μM.

7. The method of any one of claims 1-4, wherein the final concentration of the glucocorticoid analog in the solution is greater than or equal to 0.01 μM.

8. The method of any one of claims 1-4, wherein the final concentration of the glucocorticoid analog in the solution is between 0 and 1 μM.

9. The method of any one of claims 1-4, wherein the final concentration of the glucocorticoid analog in the solution is between 0 and 0.1 μM.

10. The method of any one of claims 1-4, wherein the final concentration of the glucocorticoid analog in the solution is between 0 and 0.01 μM.

11. The method of any one of claims 1-4, wherein the final concentration of the glucocorticoid analog in the solution is between 0.01 and 1 μM.

12. The method of any one of claims 1-4, wherein the final concentration of the glucocorticoid analog in the solution is between 0.01 and 0.1 μM.

13. The method of any one of claims 1-4, wherein the final concentration of the glucocorticoid analog in the solution is sufficient to produce at least 1.5-fold greater quantities of secreted rAAV compared to that produced by a host cell not contacted with a solution comprising a glucocorticoid analog.

14. The method of any one of claims 1-4, wherein the final concentration of the glucocorticoid analog in solution is sufficient to produce at least 1.5-fold greater quantities of total rAAV compared to that produced by a host cell not contacted with a solution comprising a glucocorticoid analog.

15. The method of any one of claims 1-14, wherein the host cell is contacted with the solution comprising the glucocorticoid analog for at least 2 days.

16. The method of any one of claims 1-15, further comprising the steps of harvesting and purifying the rAAV.

17. The method of any one of claims 1-16, wherein the host cell is a mammalian cell.

18. The method of claim 17, wherein the host cell is selected from the group consisting of HeLa, HEK293, COS, A549, BHK and Vero cells.

19. The method of claim 18, wherein the host cell is a HeLa cell.

20. The method of any one of claims 1-16, wherein the host cell is an insect cell.

21. The method of claim 20 wherein the host cell is selected from the group consisting of Sf9, Sf-21, Tn-368, and BTI-Tn-5B1-4 (High-Five) cells.

22. The method of any one of claims 1-21, wherein the host cell comprises a heterologous nucleotide sequence flanked by AAV inverted terminal repeats.

23. The method of any one of claims 1-22, wherein the host cell comprises rep and cap genes.

24. The method of any one of claims 1-23, wherein the host cell comprises helper virus genes.

25. The method of any one of claims 1-24, wherein the host cell comprises a heterologous nucleotide sequence flanked by AAV inverted terminal repeats, rep and cap genes, and helper virus genes.

26. The method of any one of claims 1-25, wherein the host cell produces at least 1.5-fold greater quantities of secreted rAAV compared to that produced by a host cell not contacted with a solution comprising a glucocorticoid analog.

27. The method of any one of claims 1-25, wherein the host cell produces at least 1.5-fold greater quantities of total rAAV compared to that produced by a host cell not contacted with a solution comprising a glucocorticoid analog.

28. A rAAV produced by the method of any one of claims 1-27.

29. A composition comprising the rAAV of claim 28.

30. A composition comprising a host cell and a glucocorticoid analog.
